(19) United States
(12) Patent Application Publication
(10) Pub. No.: US 2011/0004037 A1
(43) Pub. Date: Jan. 6, 2011

(54) Use of Mixed Activity Dehydrogenation Catalyst Following Oxidative Reheat

(75) Inventors: Michael A. Schultz, Kuala Lumpur (MY); Daniel A. Kauff, Arlington Heights, IL (US); Kyle D. Mankin, Prospect, KY (US)

Correspondence Address: HONEYWELL/UOP;PATENT SERVICES, 101 COLUMBIA DRIVE, P O BOX 2245 MAIL STOP AB/2B, MORRISTOWN, NJ, 07962, US

(73) Assignees: UOP LLC, Des Plaines, IL (US); SUD-CHEMIE, INCORPORATED, Louisville, KY (US)

(21) Appl. No.: 12/825,550

(22) Filed: Jun. 29, 2010

Related U.S. Application Data
(60) Provisional application No. 61/222,577, filed on Jul. 2, 2009.

(51) Int. Cl. C07C 5/327

(52) U.S. Cl. 585/319; 585/441

(57) ABSTRACT

Methods are disclosed for the dehydrogenation of feed streams, such as in the manufacture of styrene from ethylbenzene, using a catalyst bed having catalyst with differing activities. In particular, the use of upstream and downstream catalyst beds of relatively low and high activities, respectively, can reduce the production of unwanted byproducts, especially in styrene production processes employing an oxidative reheat step (oxidation zone) prior to ethylbenzene dehydrogenation. The methods allow the maximum temperature in the oxidation zone to be decreased, thereby reducing the formation of unwanted oxygenated byproducts (e.g., phenol).

### CROSS-REFERENCE TO RELATED APPLICATION

This application claims the benefit of U.S. Provisional Application No. 61/222,577 filed Jul. 2, 2009.

### FIELD OF THE INVENTION

The present invention relates to methods for the dehydrogenation of feed streams, such as in the manufacture of styrene from ethylbenzene, using catalysts with differing activities. In particular, the use of upstream and downstream catalyst beds of relatively low and high activities, respectively, can reduce the production of unwanted byproducts, especially in styrene production processes employing one or more oxidative reheat steps prior to EB dehydrogenation.

### DESCRIPTION OF RELATED ART

Styrene (phenylethylene, vinylbenzene), an important monomer used in the manufacture of many plastics, is commonly produced in a two-step process. First, ethylbenzene (EB) is formed by alkylating benzene, by transalkylating polyethylbenzenes (PEBs), or by both. The EB is then dehydrogenated, in the presence of steam (which supplies the sensible heat needed for the endothermic reaction), to produce styrene. The EB dehydrogenation effluent (or crude styrene product) is then processed in a separation section, typically using three of four distillation columns, to recover purified styrene monomer, unreacted EB and benzene, as well as byproduct benzene and other byproducts, namely toluene, and heavies (tar).

The dehydrogenation of EB to styrene is endothermic and therefore the temperature of the dehydrogenation catalyst bed decreases significantly during the progress of the reaction, thus lowering the conversion to styrene. This limitation of conversion arises at reduced temperatures, not only from the lower rate of EB dehydrogenation, but also from the lower equilibrium conversion level. Moreover, the decrease of temperature adversely affects the selectivity for styrene, because as the equilibrium conversion level is approached, essentially only undesirable side reactions continue to take place. A economically favorable level of conversion is therefore not achieved with only a single bed of dehydrogenation catalyst.

For these reasons, it has become standard commercial practice to perform interstage reheating, whereby the EB dehydrogenation effluent from a first reactor is heated to a desired inlet temperature of a second downstream reactor. A known method for maintaining the reaction temperature (referred to generally as “oxidative reheat”) is to introduce oxygen or an oxygen-containing gas which will burn or oxidize the hydrogen formed during the dehydrogenation reaction, thus increasing the temperature of the effluent stream and consequently increasing the conversion of the EB to styrene. The use of oxidative reheat is based on the recognition that the combustion of the hydrogen generated in the dehydrogenation process performs two beneficial functions. The first is a favorable shift in the dehydrogenation reaction equilibrium toward the desired dehydrogenation product as hydrogen is removed or consumed. The second is the release of heat sufficient to reheat the reactants to the desired dehydrogenation conditions, prior to a subsequent stage of dehydrogenation. Processes employing oxidative reheat utilize a hydrogen oxidation catalyst in an attempt to selectively oxidize the hydrogen rather than feed or product hydrocarbons that are also present in the dehydrogenation zone.

Various dehydrogenation processes using some form of oxidative reheat are described in the art. For instance, U.S. Pat. No. 3,437,703 discloses a dehydrogenation process which may utilize either a “homogeneous catalyst system,” in which oxidation and dehydrogenation catalysts are admixed or layered, or a “multibed” system, utilizing individual catalyst beds. Similarly, U.S. Pat. No. 3,855,330 describes a dehydrogenation process using sequential beds of dehydrogenation catalyst and oxidation catalyst. U.S. Pat. No. 3,502,737 describes an EB dehydrogenation process in which catalyst activity and stability are maintained by the careful control of the amount of oxygen which is present and by a reduction in the steam which is used in the reaction zone. The presence of oxygen is taught to prevent carbon deposits on the surface of catalytically active sites of the dehydrogenation catalyst, which is in a physical admixture with the hydrogen oxidation catalyst. U.S. Pat. No. 4,778,941 teaches the use of an eductor to withdraw a portion of a dehydrogenation effluent from the reactor, cool it, and return it to the reactor, thereby allowing for an increased amount of hydrogen to be consumed in a separate bed of oxidation catalyst.

Regardless of the particular manner in which oxidative reheat is practiced in dehydrogenation reactions such as the conversion of EB to styrene, methods are continually being sought to better promote the oxidation of hydrogen during interstage reheating, rather than the destructive combustion or oxidation of the more valuable feed and product hydrocarbons.

### SUMMARY OF THE INVENTION

Embodiments of the invention relate to the finding that the oxidation temperature in an oxidation or combustion zone, used for interstage reheating (or oxidative reheat) between successive dehydrogenation stages or zones, significantly impacts the generation of unwanted, oxidized byproducts. Importantly, methods have now been discovered whereby the oxidation zone temperature can be decreased, without adversely impacting the overall level of conversion to the desired dehydrogenation product such as styrene.

Embodiments of the invention are based on the use of dehydrogenation catalysts (e.g., EB dehydrogenation catalysts) having differing activities in upstream and downstream beds or reactor sections. The catalyst configuration, in which a more active catalyst for dehydrogenation is used downstream of a less active catalyst, allows the overall dehydrogenation process to be carried out at conditions less conducive to byproduct formation. For example, the use of catalysts with differing activities in a dehydrogenation zone downstream of an oxidation zone, as described herein, can decrease the temperature requirement (e.g., the inlet temperature, average bed temperature, and/or maximum temperature) in the upstream oxidation reaction, thereby decreasing the extent of non-selective oxidation reactions in the oxidation zone.

These and other aspects and features relating to the present invention are apparent from the following Detailed Description.

### BRIEF DESCRIPTION OF THE DRAWING

FIG. 1 shows a representative reaction section of a styrene production process, having successive dehydrogenation and oxidation zones.

### DETAILED DESCRIPTION

The present invention relates to method for dehydrogenating a feed stream comprising contacting the feed stream with a first dehydrogenation catalyst and then with a second dehydrogenation catalyst, wherein the second dehydrogenation catalyst has a higher activity than the first dehydrogenation catalyst. The first and second dehydrogenation catalysts may be in separate reactors or in separate beds within the same reactor (e.g., at inlet and outlet positions, respectively), where the beds are not necessarily physically separated, such as by the use of a distribution device or other means. The feed stream may be, for example, an oxidation zone effluent stream of an EB dehydrogenation process.

Dehydrogenation catalysts such as those used for the production of styrene from EB are known in the art and include iron oxide catalysts with promoter metals such as potassium. Methods for increasing or decreasing the dehydrogenation activity of catalysts are also known. For example, the regulation of catalytic activity can be achieved by varying the catalytic metal and promoter loadings or amounts (e.g., in percent by weight) or ratios of these components, using more or less inert material in a catalyst formulation, and/or subjecting catalyst to controlled amounts of activity attenuating agents (e.g., sulfiding agents).

Catalyst activity is generally characterized in terms of the catalyst inlet temperature or average catalyst bed temperature (e.g., the weighted average bed temperature, calculated based on temperatures measured at various axial points, which are weighted based on the catalyst weight fraction between measurement points such as those corresponding to thermocouple locations). The higher activity, second dehydrogenation catalyst described herein will generally have at least a 2° C. activity advantage over the upstream, lower activity dehydrogenation catalyst. Typically this activity advantage is in the range from about 3° C. to about 10° C. The activity advantage in this case is defined as the reduction in inlet temperature, holding all other conditions constant, required to achieve 50% conversion of a given dehydrogenation feed used in a particular process (for example 50% conversion of an EB feed to styrene).

In a representative embodiment, equal or approximately equal weights of each type of dehydrogenation catalyst are used in a dehydrogenation reactor or zone, following oxidative reheat, as discussed above. However, the downstream, higher activity dehydrogenation catalyst may generally account for at least 20% of the weight of the combined, higher and lower activity catalysts, and often accounts for about 25% to about 80% of the combined weight.

In the case where the present invention is practiced using catalysts having three or more levels of activity, for example having progressively higher activity in the downstream direction of a catalyst bed, the “higher activity catalyst” may be defined as that in any catalyst bed, which is preceded by (i.e., is positioned downstream of) a catalyst having lower activity. The relative activity advantages and relative amounts of higher and lower activity catalysts discussed above can therefore apply to any such pair of catalyst beds. In other embodiments, the catalyst activity may be gradually increased from the inlet to the outlet of the dehydrogenation reactor or zone. In this case, it is readily appreciated that the use of higher activity catalyst and lower activity catalyst may be discernable by separating the catalyst beds across a given plane or reactor cross section and considering the catalyst activity in each of the resulting, divided sections. Mixtures of dehydrogenation catalysts having differing activities, with an increasing content of the higher activity catalyst in the downstream direction of the catalyst bed, may therefore effectively be used to achieve higher and lower activity catalysts, according to the invention. In other embodiments, discreet beds, each having a single type of a higher and a lower activity catalyst, and even separate reactors employing these catalysts, may be used to achieve the advantages described herein.

Advantageously, the use of a higher, second activity dehydrogenation catalyst lowers the overall temperature needed to achieve a given EB conversion. However, it has been determined that using this higher activity dehydrogenation catalyst throughout an entire dehydrogenation reactor or reaction zone is not necessarily desirable because the presence of this catalyst at the higher reactor inlet temperature leads to undesirable byproducts, such as phenylacetylene, in the dehydrogenation reaction. Therefore, it is preferred that the higher activity catalyst be used in only a downstream portion of the dehydrogenation reactor or reaction zone.

Dehydrogenation using catalysts with differing activities is preferably carried out subsequent to, or downstream of, a selective oxidation reaction, such as the oxidative reheat step discussed above that is used for the combustion of hydrogen in a styrene production process. This beneficially reduces the temperature requirement (e.g., average catalyst bed temperature, inlet temperature, and/or maximum or outlet temperature) of the oxidation zone (e.g., the selective oxidation catalyst) without significantly affecting the conversion of the overall dehydrogenation process, such as in the production of styrene. The lower oxidation zone requirement in turn reduces the production of unwanted byproducts, and particularly oxygenated species.

It has now been found that even a relatively small reduction in the average or maximum temperature of the oxidation reaction (e.g., in an oxidation zone) can significantly decrease the formation of unwanted, oxidized byproducts formed by the reaction of oxygen with valuable feed components. For example, in the case of a typical EB dehydrogenation reactor effluent (e.g., containing styrene, unreacted EB, benzene, and light byproducts such as C4-C6 olefins and diolefins) that is subjected to interstage heating (or oxidative reheat), non-selective reactions (i.e., other than the desired combustion of hydrogen) include those which form phenol, benzaldehyde, acetophenone, benzacetaldehyde, and other oxygenates. Some of these byproducts result from the contact between the injected oxygen (typically in an oxygen-containing gas such as air) and the dehydrogenation effluent hydrocarbon mixture, in the absence of the oxidation catalyst. In any event, the reduction of the oxidation catalyst temperature decreases the relative production of these oxygenated byproducts, whether they result from thermal or catalyzed reactions.

In this manner, the use of dehydrogenation catalysts of relatively low and high activities in upstream and downstream catalyst beds, respectively, can reduce the byproduct formation in both the dehydrogenation reaction zone as well as in the prior oxidation or combustion zone, in dehydrogenation processes (e.g., in the production of styrene via EB dehydrogenation) employing interstage heating (or oxidative reheat). This reduction in byproducts not only reduces the level of contaminants in the desired product (e.g., styrene), but also improves the life of the dehydrogenation and/or oxidation catalyst, as the byproducts can deactivate either of these catalysts, possibly directly or indirectly as precursors to coke.

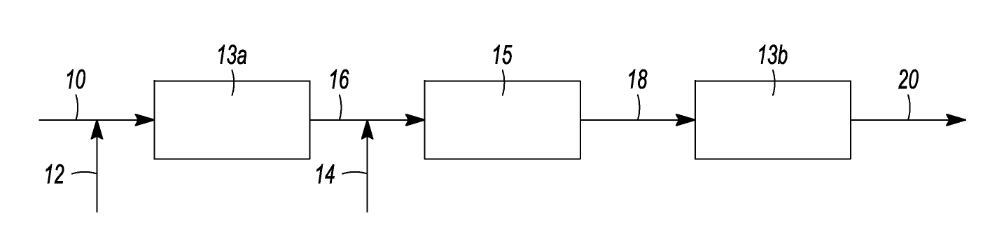

Aspects of the invention are further illustrated with reference to FIG. 1, which depicts in simplified block form the basic steps in a representative dehydrogenation process employing interstage heating between separate dehydrogenation zones. A dehydrogenation process inlet stream 10 (e.g., styrene or a mixture of styrene and EB, with the latter being present in an amount of at least 10% by weight) is contacted with a flow of steam 12 in order to carry out dehydrogenation in a first dehydrogenation zone 13a. The amount of steam used is typically in the range of 8-10 moles per mole of hydrocarbon in the inlet stream 10. After dehydrogenation to a given extent, the first dehydrogenation zone effluent 16 (e.g., an effluent stream comprising EB and hydrogen) is contacted with oxygen or an oxygen-containing stream 14 (e.g., air) to selectively combust or oxidize hydrogen produced in the first dehydrogenation zone 13a. The flow of the oxygen-containing stream 14 is typically such that about 0.05 to about 0.4 moles of oxygen are present, per mole of hydrogen in the first dehydrogenation zone effluent 16. The operating pressure of the oxidation zone 15 is normally near atmospheric pressure, although somewhat higher or lower (e.g., subatmospheric) pressures may be used.

The first dehydrogenation zone effluent 16, due to the endothermic nature of the preceding dehydrogenation reaction, is normally at a temperature from about 500-550° C. (about 930-1020° F.). The use of the oxidation zone 15, by virtue of the highly exothermic combustion reaction, raises the temperature of the oxidation zone effluent 18 generally to about 600-650° C. (about 1110-1200° F.), which normally approximates the temperature of the dehydrogenation process inlet stream 10, flowing to the first dehydrogenation zone 13a. The rate of oxygen injection can be controlled to achieve a desired temperature of the oxidation zone effluent 18, or a desired temperature increase from the first dehydrogenation zone effluent 16 to the oxidation zone effluent 18.

Oxidation therefore occurs in the oxidation zone 15 to provide an oxidation zone effluent stream (e.g., comprising EB) 18, which then serves as a feed stream to a second dehydrogenation zone 13b. The use of first and second dehydrogenation catalysts with differing activities, as discussed above, is most advantageously used in this second dehydrogenation zone 13b. Importantly, this dual catalyst configuration allows for a reduction in the temperature of upstream oxidation zone effluent stream 18, without sacrificing dehydrogenation conversion, as measured based on the composition of the second dehydrogenation zone effluent 20. In fact, this temperature has been found to be largely dependent on the activity of the dehydrogenation catalyst in the second dehydrogenation zone. Because a relatively more active dehydrogenation catalyst is used in an outlet position, bed, or portion of this zone or reactor, a lower inlet temperature of the oxidation zone effluent stream 18 is possible. This allows the average and/or maximum temperature at which oxygen and hydrocarbons are contacted in the oxidation zone to be reduced, thereby significantly hindering the formation of detrimental byproducts (e.g., oxygenated compounds as discussed above). The second dehydrogenation zone effluent 20 may represent the final dehydrogenation reactor effluent which may be routed, for example, to a downstream separation section. Otherwise, this stream may be subjected to additional oxidative reheat in another oxidation zone, followed by another dehydrogenation zone.

The use of the second, more active dehydrogenation catalyst throughout the second dehydrogenation zone 13b is avoided, in order to prevent the formation of excessive dehydrogenation byproducts, such as phenylacetylene, at the higher temperature inlet of this dehydrogenation zone. Therefore, the dehydrogenation processes described herein and employing dual activity catalysts downstream of an oxidative reheat zone have a number of possible advantages. As discussed above, byproduct formation in both the oxidation and dehydrogenation zones can be reduced, the life of the dehydrogenation catalyst extended, and the product quality improved. Moreover, because a lower temperature is used for the feed stream to the dehydrogenation zone (e.g., the second dehydrogenation zone 13b, as depicted in FIG. 1), a reduced amount of oxygen is required in the upstream oxidation zone 15, providing a cost savings.

Overall, aspects of the invention are directed to the use of catalysts having differing levels of activity, with a higher activity catalyst being positioned downstream with respect to a lower activity catalyst, in dehydrogenation processes such as in the dehydrogenation of EB to produce styrene. In view of the present disclosure, it will be seen that several advantages may be achieved and other advantageous results may be obtained. Those having skill in the art will recognize the applicability of the methods disclosed herein to any of a number of dehydrogenation processes in which oxidative reheat may be currently employed or contemplated, including the dehydrogenation of butane and other paraffins, such as any of those having carbon numbers in the C3-C14 range. It will also become apparent that embodiments of the methods disclosed herein will use multiple catalyst beds with varying activities, or catalyst beds having varying activity with respect to the axial bed position, to achieve any or all of the advantages described herein.

Those having skill in the art, with the knowledge gained from the present disclosure, will recognize that various changes could be made in the above processes without departing from the scope of the present disclosure. Mechanisms used to explain theoretical or observed phenomena or results, shall be interpreted as illustrative only and not limiting in any way the scope of the appended claims.

The following examples are set forth as representative of the present invention. These examples are not to be construed as limiting the scope of the invention as these and other equivalent embodiments will be apparent in view of the present disclosure and appended claims.

### Example 1

### Effect of Oxidation Temperature on Byproduct Formation Using a Model Feed

The combined air/hydrocarbon/water inlet stream to an interstage oxidative reheat zone of a commercial styrene production process was modeled using a stream that combined the following flows: (1) air at 802 standard cc per minute, (2) hydrocarbon (67% styrene and 33% EB, by weight) at 140 grams/hr, and (3) water at 265 grams/hr.

The effect of oxidation temperature on oxygenate formation was studied and the results in the following table were obtained:

| Oxidation Temperature (° C.) | Total Oxygenates (wt. %) |
|---|---|
| 250 | 0.014 |
| 510 | 0.097 |
| 550 | 0.233 |
| 590 | 0.356 |

The above results show the importance of minimizing the temperature at which oxygen is in contact with hydrocarbon. Because the oxidation reaction is exothermic, such that the temperature within the oxidation reactor or reaction zone can increase by as much as 100° C., the oxygen is in contact with hydrocarbon at a range of temperatures throughout oxidation. Oxygenate byproduct formation increases significantly with increasing temperature, as shown by the results obtained above. The methods disclosed herein can beneficially reduce the oxidation temperature to lower byproduct formation and thereby improve catalyst life and product quality.

1. A method for dehydrogenating a feed stream comprising contacting the feed stream with a first dehydrogenation catalyst and then with a second dehydrogenation catalyst, wherein the second dehydrogenation catalyst has a higher activity than the first dehydrogenation catalyst.

2. The method of claim 1, wherein the second dehydrogenation catalyst has at least a 2° C. activity advantage over the first dehydrogenation catalyst.

3. The method of claim 1, wherein the activity advantage is in the range from about 3° C. to about 10° C.

4. The method of claim 1, wherein the second dehydrogenation catalyst accounts for at least 20% of the weight of the combined, first and second dehydrogenation catalysts.

5. The method of claim 4, wherein the second dehydrogenation catalyst accounts for from about 25% to about 80% of the combined weight of the combined first and second dehydrogenation catalysts.

6. The method of claim 1, wherein the feed stream is an oxidation zone effluent stream of an ethylbenzene (EB) dehydrogenation process.

7. The method of claim 1, wherein the first and second dehydrogenation catalysts are at inlet and outlet positions, respectively, within a single reactor.

8. A method for dehydrogenating ethylbenzene (EB) to produce styrene, the method comprising:
(a) oxidizing, in an oxidation zone, an effluent stream comprising EB and hydrogen from a first dehydrogenation reaction zone, to provide an oxidation zone effluent stream comprising EB, and
(b) dehydrogenating EB in the oxidation zone effluent stream with a first dehydrogenation catalyst and then with a second dehydrogenation catalyst, wherein the second dehydrogenation catalyst has a higher activity than the first dehydrogenation catalyst.

9. The method of claim 8, wherein oxidizing in step (a) is carried out in the presence of an oxygen-containing gas.

10. The method of claim 9, wherein the oxygen-containing gas is air.
